(19) United States
(12) Patent Application Publication
(10) Pub. No.: US 2020/0323488 A1
(43) Pub. Date: Oct. 15, 2020

(54) A METHOD AND AN APPARATUS FOR PRODUCING INFORMATION INDICATIVE OF METABOLIC STATE

(71) Applicant: Lappeenrannan-Lahden teknillinen yliopisto LUT, LAPPEENRANTA (FI)

(72) Inventors: Mikko KUISMA, LAPPEENRANTA (FI); Antti IMMONEN, LAPPEENRANTA (FI); Saku LEVIKARI, LAPPEENRANTA (FI)

(21) Appl. No.: 16/753,508

(22) PCT Filed: Aug. 21, 2018
(86) PCT No.: PCT/FI2018/050590

§ 371 Date: Apr. 3, 2020

(30) Foreign Application Priority Data
Oct. 4, 2017 (FI) 20175875

(51) Int. Cl. A61B 5/00; A61B 5/01; A61B 5/0205

(52) U.S. Cl. CPC A61B 5/4866; A61B 5/01; A61B 5/024; A61B 5/7278; A61B 5/02055

(57) ABSTRACT

An apparatus for producing information indicative of metabolic state of a metabolic energy system comprises a processing device for receiving a signal that is indicative of a heat-flux generated by the metabolic energy system. The processing device is configured to maintain model data expressing relative contributions of the phosphagen system, the glycolytic system, and the aerobic system to muscular energy production as functions of time during physical loading of the metabolic energy system. The processing device is configured to form estimates for the energy production of the phosphagen system, the energy production of the glycolytic system, and the energy production of the aerobic system as functions of time and based on the model data and the signal indicative of the heat-flux. The estimates can be indicative of the instant metabolic state, and they can be utilized in physical training, weight control, and detection of metabolism-related health issues.

### TECHNICAL FIELD

The disclosure relates to a method and an apparatus for producing information indicative of metabolic state of a metabolic energy system. Furthermore, the disclosure relates to a computer program for producing information indicative of metabolic state of a metabolic energy system.

### BACKGROUND

Muscular metabolic energy production can be separated in three main systems which are related to activities of different intensities and durations. The phosphagen system, i.e. the adenosine triphosphate-creatine phosphate “ATP-CP”, supports brief and high-intensity activities having durations of few seconds. The glycolytic system provides energy for activities of longer durations and lower intensities. The durations of activities energized by the glycolytic system are typically tens of seconds. The aerobic system, i.e. the oxidative system, supports long-duration, lower-intensity activities like distance running. The durations of activities exceeding the basal metabolic rate and energized by the aerobic system can be several hours.

In many situations, there is a need for information indicative of metabolic state during a physical exercise. For example, with the aid of such information, a person can train at an optimal level giving better results for example in sports, fitness, healthcare, and/or weight control. Furthermore, the information about the metabolic state facilitates maximizing training effectiveness and preventing overtraining and fatigues. Typical devices for producing information indicative of metabolic energy production are heart-beat rate sensors, pedometers, electromyographical “EMG” sensors, instruments for measuring respiratory gas exchange i.e. oxygen intake and CO₂ production, calorimetric instruments, and means for measuring lactate from blood. An inconvenience related to many devices for estimating metabolic energy production is that they do not provide information about instant metabolic energy production but only a time-average of the metabolic energy production so that one cannot see e.g. a current trend of the metabolic state. An inconvenience related to some devices, e.g. instruments for measuring respiratory gas exchange, is that they require a complex instrumentation and thus they are not suitable for being a small portable device.

Direct energy measurement based on a heat-flux sensor has been used in commercial products, e.g. LifeChek™. However, many available products measure only an average of a long-term energy production and thus they do not produce information indicative of the instant metabolic state during a physical exercise.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In accordance with the invention, there is provided a new apparatus for producing information indicative of metabolic state of a metabolic energy system. An apparatus according to the invention comprises:
a signal interface for receiving a signal indicative of a heat-flux generated by a metabolic energy system, and
a processing device coupled to the signal interface and configured to:
   maintain model data expressing relative contributions of the phosphagen system, the glycolytic system, and the aerobic system to muscular energy production as functions of time during physical loading of the metabolic energy system, and
   form an estimate for energy production of the phosphagen system, an estimate for energy production of the glycolytic system, and an estimate for energy production of the aerobic system as functions of time and based on the model data and the signal indicative of the heat-flux.

The above-mentioned estimates can be indicative of the instant metabolic state of the metabolic energy system. The estimates can be utilized for example in physical training, weight control, and detection of metabolism-related health issues such as e.g. diabetes. The estimates make it easier to maximize training effectiveness and prevent overtraining and fatigues. Furthermore, the above-mentioned estimates facilitate monitoring recovery, avoiding lactic acidocis, and detecting metabolic disorders.

The above-described apparatus may further comprise a heat-flux sensor for measuring the heat-flux. It is also possible that the signal interface is suitable for receiving a signal from an external heat-flux sensor, i.e. it is emphasized that the apparatus does not necessarily comprise any heat-flux sensor for measuring the heat-flux. It is also possible that the signal interface is suitable for receiving signals from many heat-flux sensors. In this exemplifying case, the apparatus may comprise many heat-flux sensors or the signal interface is suitable for receiving signals from many external heat-flux sensors. The above-described apparatus can be a portable device and each heat-flux sensor can be placed on e.g. a wrist band, a chest band, a strap, a belt, or another wearable item.

In accordance with the invention, there is provided also a new method for producing information indicative of metabolic state of a metabolic energy system. A method according to the invention comprises:
receiving a signal indicative of a heat-flux generated by the metabolic energy system,
maintaining model data expressing relative contributions of the phosphagen system, the glycolytic system, and the aerobic system to muscular energy production as functions of time during physical loading of the metabolic energy system, and
forming an estimate for the energy production of the phosphagen system, an estimate for the energy production of the glycolytic system, and an estimate for the energy production of the aerobic system as functions of time and based on the model data and the signal indicative of the heat-flux.

In accordance with the invention, there is provided also a new computer program for producing information indicative of metabolic state of a metabolic energy system. A computer program according to the invention comprises computer executable instructions for controlling a programmable processor to:
receive a signal indicative of a heat-flux generated by the metabolic energy system,
maintain model data expressing relative contributions of the phosphagen system, the glycolytic system, and the aerobic system to muscular energy production as functions of time during physical loading of the metabolic energy system, and
form an estimate for the energy production of the phosphagen system, an estimate for the energy production of the glycolytic system, and an estimate for the energy production of the aerobic system as functions of time and based on the model data and the signal indicative of the heat-flux.

In accordance with the invention, there is provided also a new computer program product. The computer program product comprises a non-volatile computer readable medium, e.g. a compact disc “CD”, encoded with a computer program according to the invention.

Various exemplifying and non-limiting embodiments of the invention are described in accompanied dependent claims.

Various exemplifying and non-limiting embodiments of the invention both as to constructions and to methods of operation, together with additional objects and advantages thereof, will be best understood from the following description of specific exemplifying and non-limiting embodiments when read in conjunction with the accompanying drawings.

The verbs “to comprise” and “to include” are used in this document as open limitations that neither exclude nor require the existence of unrecited features. The features recited in dependent claims are mutually freely combinable unless otherwise explicitly stated. Furthermore, it is to be understood that the use of “a” or “an”, i.e. a singular form, throughout this document does not exclude a plurality.

### BRIEF DESCRIPTION OF THE FIGURES

Exemplifying and non-limiting embodiments of the invention and their advantages are explained in greater detail below in the sense of examples and with reference to the accompanying drawings, in which:

FIG. 1a shows a flowchart of a method according to an exemplifying and non-limiting embodiment of the invention for producing information indicative of metabolic state of a metabolic energy system,

FIG. 1b shows a schematic illustration of exemplifying model data expressing relative contributions of the phosphagen system, the glycolytic system, and the aerobic system to muscular energy production as functions of time during physical loading of a metabolic energy system,

FIG. 2 illustrates schematically an apparatus according to an exemplifying and non-limiting embodiment of the invention, and

FIG. 3 illustrates schematically an apparatus according to another exemplifying and non-limiting embodiment of the invention.

### DESCRIPTION OF THE EXEMPLIFYING EMBODIMENTS

The specific examples provided in the description below should not be construed as limiting the scope and/or the applicability of the accompanied claims. Lists and groups of examples provided in the description are not exhaustive unless otherwise explicitly stated.

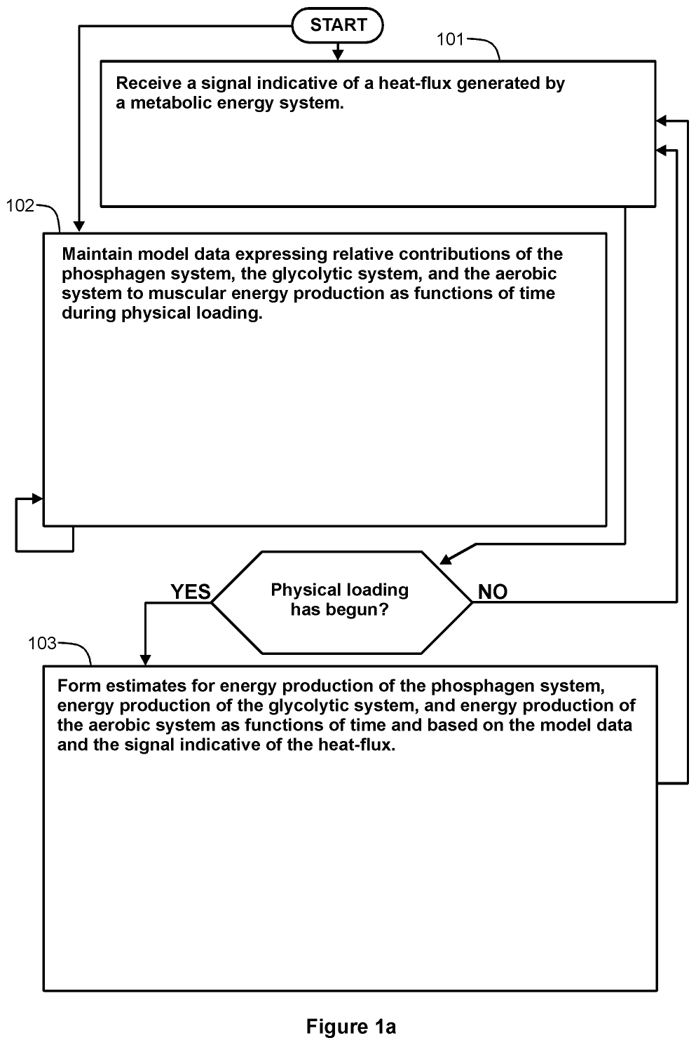

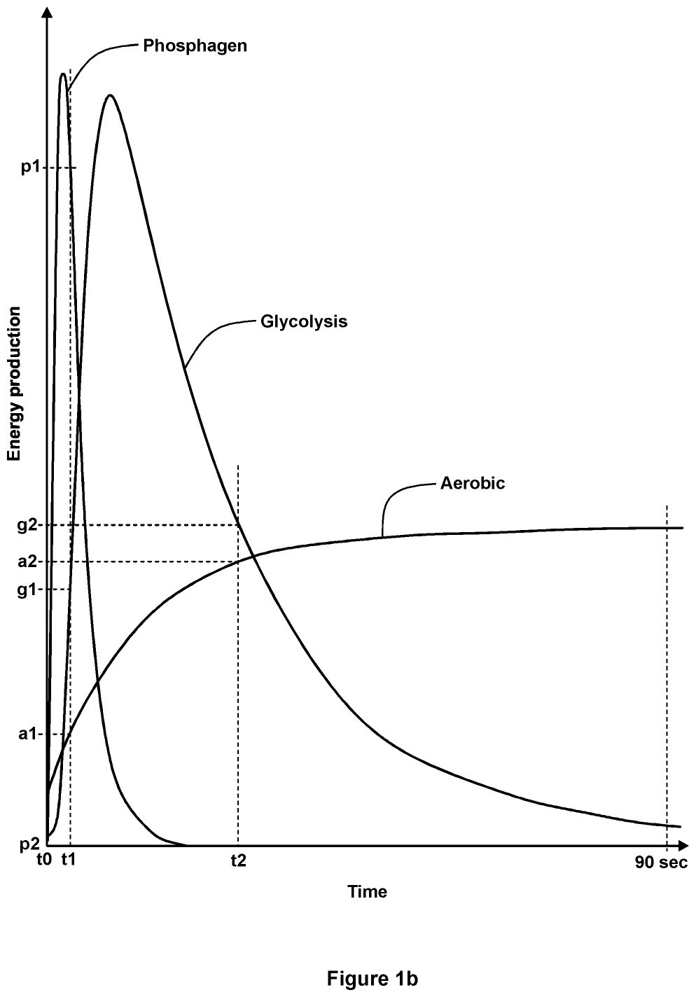

FIG. 1a shows a flowchart of a method according to an exemplifying and non-limiting embodiment of the invention for producing information indicative of metabolic state of a metabolic energy system. The method comprises in phase 101: receiving a signal indicative of a heat-flux generated by the metabolic energy system. The heat-flux is measured with a heat-flux sensor on a human or animal body which represents the metabolic energy system under consideration. The method comprises in phase 102: maintaining model data that expresses relative contributions of the phosphagen system, the glycolytic system, and the aerobic system to muscular energy production as functions of time during physical loading of the metabolic energy system. In FIG. 1b, exemplifying model data is depicted with curves which express the relative contributions of the phosphagen system, the glycolytic system, and the aerobic system as functions of time during physical loading that has begun at a moment of time to. The exemplifying curves shown in FIG. 1b correspond to an all-out exercise having a duration of 90 seconds. The method comprises in phase 103: forming an estimate for the energy production of the phosphagen system, an estimate for the energy production of the glycolytic system, and an estimate for the energy production of the aerobic system as functions of time and based on the model data and the signal indicative of the heat-flux. An exemplifying way to form the above-mentioned estimates for two exemplifying moments of time t1 and t2 is presented below.

In this exemplifying case, the signal indicative of the measured heat-flux is assumed to be S1 at the moment of time t1 and S2 at the moment of time t2. The total muscular energy production is assumed to be directly proportional to the measured heat-flux. Thus, the total muscular energy production is α×S1 at the moment of time t1, and correspondingly the total muscular energy production is α×S2 at the moment of time t2, where a is a constant ratio between the total muscular energy production and the measured heat-flux. It is however also possible to use a more complex and sophisticated conversion rule between the total muscular energy production and the signal indicative of the measured heat-flux. The unit of the heat-flux and the muscular energy production can be e.g. Watt, i.e. Joule/second.

As shown in FIG. 1b, the ratios of the contributions of the phosphagen system, the glycolytic system, and the aerobic system are p1:g1:a1 at the moment of time t1. Thus, the estimate Ep1 for the energy production of the phosphagen system at the moment of time t1 is:

Ep1=α×S1×p1/(p1+g1+a1). (1)

The estimate Eg1 for the energy production of the glycolytic system at the moment of time t1 is:

Eg1=α×S1×g1/(p1+g1+a1). (2)

The estimate Ea1 for the energy production of the aerobic system at the moment of time t1 is:

Ea1=α×S1×a1/(p1+g1+a1). (3)

Correspondingly, the estimates for the energy production of the phosphagen system, the energy production of the glycolytic system, and the energy production of the aerobic system at the moment of time t2 are:

Ep2=α×S2×p2/(p2+g2+a2), (4)

Eg2=α×S2×g2/(p2+g2+a2), and (5)

Ea2=α×S2×a2/(p2+g2+a2). (6)

As illustrated by the above-presented examples, the estimates for the energy production of the phosphagen system, the energy production of the glycolytic system, and the energy production of the aerobic system can be obtained at an arbitrary moment of time. The estimates can be formed nearly in real-time since the heat-flux generated by the metabolic system follows the instant metabolic state with a short response time and a heat-flux sensor can be selected so that the signal indicative of the heat-flux follows the real heat-flux with a short response time. Therefore, the estimates are indicative of the instant state of the metabolic energy system. The estimates can be utilized for example in physical training, weight control, and detection of metabolism-related health issues such as e.g. diabetes. The estimates make it easier to maximize training effectiveness and prevent overtraining and fatigues. Furthermore, the above-mentioned estimates facilitate monitoring recovery, avoiding lactic acidocis, and detecting metabolic disorders. It is, however, also possible that the estimates are formed off-line based on the model data and recorded values of the signal indicative of the measured heat-flux. FIG. 1 corresponds to an exemplifying case where the estimates are formed nearly in real-time.

The model data is typically person-specific, i.e. the curves shown in FIG. 1b are typically person-specific. For example, the long-term level of the total muscular energy production of an endurance trained person is typically higher than that of a sprint trained person whereas the momentary maximum of the total muscular energy production of a sprint trained person is typically higher than that of an endurance trained person. Thus, the ratio of the maximum of the Phosphagen-curve shown in FIG. 1b to the maximum of the Aerobic-curve is typically higher in conjunction with a sprint trained person than in conjunction with an endurance trained person.

The accuracy of the above-mentioned estimates can be improved with additional measurements on the human or animal body representing the metabolic energy system under consideration. A method according to an exemplifying and non-limiting embodiment of the invention comprises receiving a heart-beat rate signal indicative of a heart-beat rate. The method comprises increasing the estimate of the energy production of the aerobic system and decreasing the estimates of the energy productions of the phosphagen system and the glycolytic system in response to an increase of the heart-beat rate. This approach is based on an assumption that the increase of the heart-beat rate indicates that the relative share of the aerobic energy production increases with respect to the energy productions of the phosphagen system and the glycolytic system. The rule how the increase of the heart-beat rate is taken into account can be based on e.g. empirical data. For another example, the heart-beat rate signal can be used for correcting the relation between the total muscular energy production and the signal indicative of the measured heat-flux. The correction rule can be based on e.g. empirical data.

A method according to an exemplifying and non-limiting embodiment of the invention comprises receiving an acceleration signal. The acceleration signal can be used for example detecting the beginning of the physical loading, i.e. for detecting the time moment t0 shown in FIG. 1b. For another example, the acceleration signal can be used for correcting the relation between the total muscular energy production and the signal indicative of the measured heat-flux. The correction rule can be based on e.g. empirical data. An acceleration sensor can be attached on e.g. a limb of a person under consideration.

A method according to an exemplifying and non-limiting embodiment of the invention comprises receiving an electromyography “EMG” signal. The EMG-signal can be used for example detecting the beginning of the physical loading. For another example, the EMG-signal can be used for correcting the relation between the total muscular energy production and the signal indicative of the measured heat-flux. The correction rule can be based on e.g. empirical data. An EMG-sensor can be attached on e.g. a limb of a person under consideration.

A computer program according to an exemplifying and non-limiting embodiment of the invention comprises computer executable instructions for controlling a programmable processor to carry out actions related to a method according to any of the above-described exemplifying embodiments of the invention.

A computer program according to an exemplifying and non-limiting embodiment of the invention comprises software modules for producing information indicative of metabolic state of a metabolic energy system. The software modules comprise computer executable instructions for controlling a programmable processor to:
receive a signal indicative of a heat-flux generated by the metabolic energy system,
maintain model data expressing relative contributions of the phosphagen system, the glycolytic system, and the aerobic system to muscular energy production as functions of time during physical loading of the metabolic energy system, and
form an estimate for the energy production of the phosphagen system, an estimate for the energy production of the glycolytic system, and an estimate for the energy production of the aerobic system as functions of time and based on the model data and the signal indicative of the heat-flux.

The above-mentioned software modules can be e.g. subroutines or functions implemented with a suitable programming language.

A computer program product according to an exemplifying and non-limiting embodiment of the invention comprises a computer readable medium, e.g. a compact disc “CD”, encoded with a computer program according to an embodiment of invention.

A signal according to an exemplifying and non-limiting embodiment of the invention is encoded to carry information defining a computer program according to an embodiment of invention. In this exemplifying case, the computer program can be downloadable from a server that may constitute e.g. a part of a cloud service.

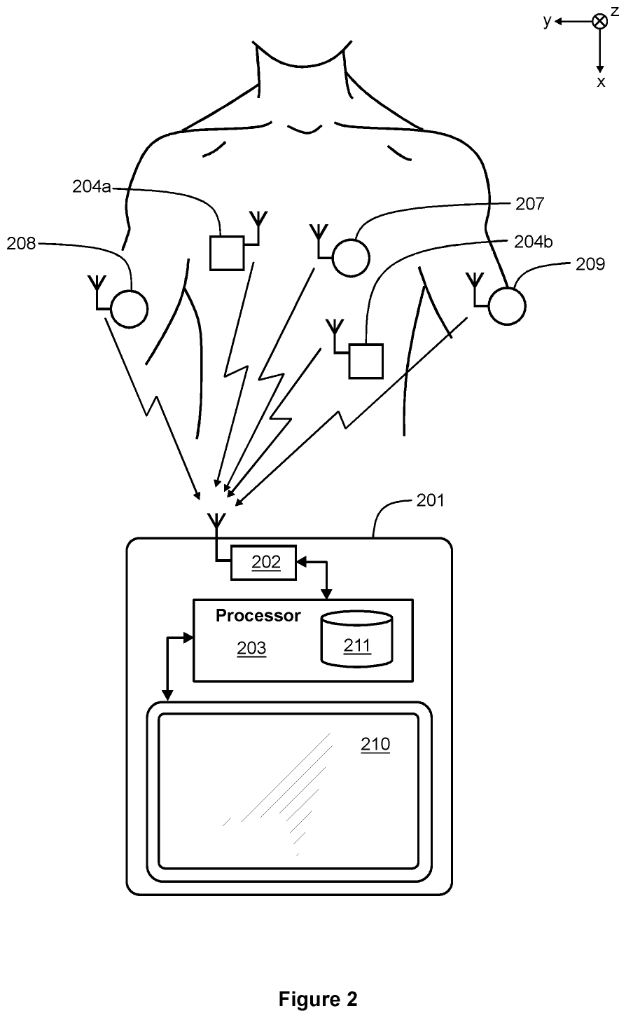

FIG. 2 illustrates schematically an apparatus 201 according to an exemplifying and non-limiting embodiment of the invention. The apparatus 201 comprises a signal interface 202 for receiving a signal indicative of a heat-flux generated by a metabolic energy system. In the exemplifying situation shown in FIG. 2, a human body represents the metabolic energy system. In the exemplifying apparatus 201 illustrated in FIG. 2, the signal interface 202 comprises a short-range radio receiver for receiving a radio signal from a heat-flux sensor 204a that comprises a short-range radio transmitter. The heat-flux sensor 204a can be based on for example multiple thermoelectric junctions so that tens, hundreds, or even thousands of thermoelectric junctions are connected in series. For another example, the heat-flux sensor 206 can be based on one or more anisotropic elements where electromotive force is created from a heat-flux by the Seebeck effect. The anisotropy can be implemented with suitable anisotropic material such as for example single-crystal bismuth. Another option for implementing the anisotropy is a multilayer structure where layers are oblique with respect to a surface of the heat-flux sensor for receiving the heat-flux. For a third example, the heat-flux sensor 204a can be based on a contact junction between pieces of different materials so that a first one of the pieces that is nearer to a human or animal body is significantly smaller in mass and heat capacity than the other one of the pieces. Thus, a heat-flux from a human or animal body causes a temperature difference from the first piece to the second piece but no significant temperature increase in the second piece. Therefore, an electromotive force caused by the temperature difference in the contact junction is indicative of the heat-flux. In an apparatus according to an exemplifying and non-limiting embodiment of the invention, the signal interface 202 is suitable for receiving signals from many heat-flux sensors, e.g. from the heat-flux sensor 204a and from a heat-flux sensor 204b, too.

The apparatus 201 comprises a processing device 203 coupled to the signal interface 202. The processing device 203 is configured to maintain model data that expresses relative contributions of the phosphagen system, the glycolytic system, and the aerobic system to muscular energy production as functions of time during physical loading of the metabolic energy system. Exemplifying model data is depicted with curves in FIG. 1b. The processing device 203 is configured to form an estimate for energy production of the phosphagen system, an estimate for energy production of the glycolytic system, and an estimate for energy production of the aerobic system as functions of time and based on the model data and the signal indicative of the heat-flux.

In an apparatus according to an exemplifying and non-limiting embodiment of the invention, the processing device 203 is configured to receive a heart-beat rate signal indicative of a heart-beat rate from a heart-beat rate sensor 207. The processing device 203 can be configured to increase the estimate of the energy production of the aerobic system and decrease the estimates of the energy productions of the phosphagen system and the glycolytic system in response to an increase of the heart-beat rate. The rule how the increase of the heart-beat rate is taken into account can be based on e.g. empirical data. For another example, the heart-beat rate signal can be used for correcting the relation between the total muscular energy production and the signal indicative of the measured heat-flux. The correction rule can be based on e.g. empirical data.

In an apparatus according to an exemplifying and non-limiting embodiment of the invention, the processing device 203 is configured to receive an acceleration signal from an acceleration sensor 208. The processing device 203 can be configured to detect the beginning of the physical loading based on the acceleration signal, i.e. to detect the time moment t0 shown in FIG. 1b. For another example, the acceleration signal can be used for correcting the relation between the total muscular energy production and the signal indicative of the measured heat-flux. The correction rule can be based on e.g. empirical data.

In an apparatus according to an exemplifying and non-limiting embodiment of the invention, the processing device 203 is configured to receive an electromyography “EMG” signal from an EMG-sensor 209. The processing device 203 can be configured to detect the beginning of the physical loading based on the EMG-signal. For another example, the EMG-signal can be used for correcting the relation between the total muscular energy production and the signal indicative of the measured heat-flux. The correction rule can be based on e.g. empirical data.

In an apparatus according to an exemplifying and non-limiting embodiment of the invention, the processing device 203 is provided with a signal input for receiving a trigger signal which is operated e.g. manually and which indicates the beginning of the physical loading, i.e. the time moment t0 shown in FIG. 1b.

In the exemplifying case illustrated in FIG. 2, the apparatus 201 comprises a user interface 210 that can be for example a touch screen.

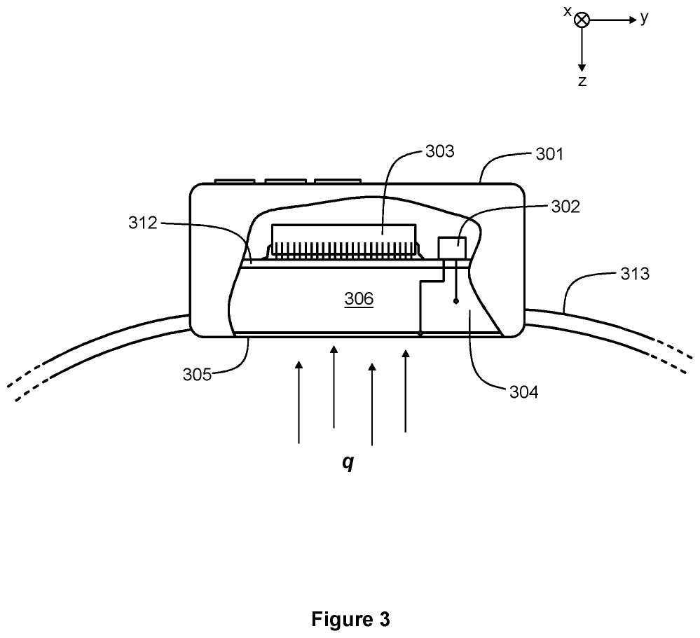

FIG. 3 illustrates schematically an apparatus 301 according to an exemplifying and non-limiting embodiment of the invention. In this exemplifying case, the apparatus 301 is a portable device which comprises a fastening band 313 that can be for example a wrist band, a chest band, a strap, or a belt. In FIG. 3, the casing of the apparatus 301 is presented as partially open cut so as to illustrate the elements inside the casing. The apparatus 301 comprises a heat-flux sensor 304 for producing a signal indicative of a heat-flux q received from a human or animal body. The apparatus 301 comprises a signal interface 302 for receiving the signal from the heat-flux sensor 304 and for converting the signal into a form suitable for a processing device 303 of the apparatus 301. The signal interface 302 may comprise for example an analog-to-digital converter “ADC”. In this exemplifying case, the heat-flux sensor 304 is based on a contact junction between pieces of different materials so that a first piece 305 that is nearer to the human or animal body is significantly smaller in mass and heat capacity than a second piece 306. Thus, the heat-flux q causes a temperature difference from the first piece 305 to the second piece 306 but no significant temperature increase in the second piece 306. Therefore, an electromotive force caused by the temperature difference in the contact junction is indicative of the heat-flux q. The heat-flux sensor 304 further comprises a first electric conductor from the first piece 305 to the signal interface 302, and a second electric conductor from the second piece 306 to the signal interface 302. The first piece 305 can be made of for example aluminum, copper, molybdenum, constantan, or nichrome. The second piece 306 can be made of for example steel, aluminum, copper, molybdenum, constantan, or nichrome. The materials of the first and second pieces 305 and 306 are advantageously chosen so that the materials are thermoelectrically dissimilar to maximize the generation of the electromotive force. In the exemplifying heat-flux sensor 304 illustrated in FIG. 3, the first piece 305 is a thin material sheet on a surface of the second piece 306. The thickness of the material sheet can be e.g. from 0.001 mm to 1 mm. Therefore, the mass of the second piece 306 can be hundreds or even thousands of times the mass of the first piece 305. In this exemplifying case, the apparatus 301 further comprises a circuit board 312 on which the processing device 303 and the signal interface 302 are mounted.

The processing device 303 of the apparatus 301 is configured to maintain model data that expresses relative contributions of the phosphagen system, the glycolytic system, and the aerobic system to muscular energy production as functions of time during physical loading of a human or animal body. The processing device 303 is configured to form an estimate for energy production of the phosphagen system, an estimate for energy production of the glycolytic system, and an estimate for energy production of the aerobic system as functions of time and based on the model data and the signal indicative of the heat-flux.

The processing device 203 of the apparatus 201 illustrated in FIG. 2 as well as the processing device 303 of the apparatus 301 illustrated in FIG. 3 can be, for example, implemented with one or more processor circuits, each of which can be a programmable processor circuit provided with appropriate software, a dedicated hardware processor such as, for example, an application specific integrated circuit “ASIC”, or a configurable hardware processor such as, for example, a field programmable gate array “FPGA”. Furthermore, the processing device 203 may comprise memory 211 which can be e.g. random-access memory “RAM”. Correspondingly, the apparatus 301 may comprise one or more memory circuits separate from the processing device 303 and/or the processing device 303 may comprise integrated memory.

The specific examples provided in the description given above should not be construed as limiting the applicability and/or interpretation of the appended claims. It is to be noted that lists and groups of examples given in this document are non-exhaustive lists and groups unless otherwise explicitly stated.

1. An apparatus comprising:
a signal interface for receiving a signal indicative of a heat-flux generated by a metabolic energy system, and
a processing device coupled to the signal interface,
wherein the processing device is configured to:
maintain model data expressing relative contributions of a phosphagen system, a glycolytic system, and an aerobic system to muscular energy production as functions of time during physical loading of the metabolic energy system, and
form an estimate for energy production of the phosphagen system, an estimate for energy production of the glycolytic system, and an estimate for energy production of the aerobic system as functions of time and based on the model data and the signal indicative of the heat-flux.

2. An apparatus according to claim 1, wherein the apparatus further comprises a heat-flux sensor for measuring the signal on a human or animal body, the heat-flux sensor being connected to the signal interface.

3. An apparatus according to claim 2, wherein the heat-flux sensor comprises:
first and second pieces made of different materials and arranged to constitute a contact junction of the materials for generating electromotive force in response to a temperature difference between the first and second pieces, and
a first electric conductor connected to the first piece and a second electric conductor connected to the second piece, the electromotive force being detectable from between ends of the first and second electric conductors,
wherein a mass and a heat capacity of the second piece are greater than a mass and a heat capacity of the first piece so that the temperature difference between the first and second pieces caused by the heat-flux across the contact junction from the first piece to the second piece is greater than a temperature increase caused by the heat-flux at a point of the second piece where the second electric conductor is connected to the second piece.

4. An apparatus according to claim 3, wherein the mass of the second piece is at least one hundred times the mass of the first piece.

5. An apparatus according to claim 1, wherein the processing device is configured to receive a heart-beat rate signal indicative of a heart-beat rate and to increase the estimate of the energy production of the aerobic system and decrease the estimates of the energy productions of the phosphagen system and the glycolytic system in response to an increase of the heart-beat rate.

6. An apparatus according to claim 1, wherein the processing device is configured to receive an acceleration signal and to detect a beginning of the physical loading based on the acceleration signal.

7. An apparatus according to claim 1, wherein the processing device is configured to receive an electromyography signal and to detect a beginning of the physical loading based on the electromyography signal.

8. A method for producing information indicative of metabolic state of a metabolic energy system, the method comprising:
receiving a signal indicative of a heat-flux generated by the metabolic energy system,
maintaining model data expressing relative contributions of a phosphagen system, a glycolytic system, and an aerobic system to muscular energy production as functions of time during physical loading of the metabolic energy system, and
forming an estimate for energy production of the phosphagen system, an estimate for energy production of the glycolytic system, and an estimate for energy production of the aerobic system as functions of time and based on the model data and the signal indicative of the heat-flux.

9. A method according to claim 8, wherein the method comprises measuring the heat-flux on a human or animal body representing the metabolic energy system.

10. A method according claim 8, wherein the method comprises receiving a heart-beat rate signal indicative of a heart-beat rate, and increasing the estimate of the energy production of the aerobic system and decreasing the estimates of the energy productions of the phosphagen system and the glycolytic system in response to an increase of the heart-beat rate.

11. A method according to claim 8, wherein the method comprises receiving an acceleration signal and detecting a beginning of the physical loading based on the acceleration signal.

12. A method according to claim 8, wherein the method comprises receiving an electromyography signal and detecting a beginning of the physical loading based on the electromyography signal.

13. A non-transitory computer readable medium encoded with a computer program for producing information indicative of metabolic state of a metabolic energy system, the computer program comprising computer executable instructions for controlling a programmable processor to:
receive a signal indicative of a heat-flux generated by the metabolic energy system,
maintain model data expressing relative contributions of a phosphagen system, a glycolytic system, and an aerobic system to muscular energy production as functions of time during physical loading of the metabolic energy system, and
form an estimate for energy production of the phosphagen system, an estimate for energy production of the glycolytic system, and an estimate for energy production of the aerobic system as functions of time and based on the model data and the signal indicative of the heat-flux.

14. (canceled)

15. A method according claim 9, wherein the method comprises receiving a heart-beat rate signal indicative of a heart-beat rate, and increasing the estimate of the energy production of the aerobic system and decreasing the estimates of the energy productions of the phosphagen system and the glycolytic system in response to an increase of the heart-beat rate.

16. An apparatus according to claim 2, wherein the processing device is configured to receive a heart-beat rate signal indicative of a heart-beat rate and to increase the estimate of the energy production of the aerobic system and decrease the estimates of the energy productions of the phosphagen system and the glycolytic system in response to an increase of the heart-beat rate.

17. An apparatus according to claim 3, wherein the processing device is configured to receive a heart-beat rate signal indicative of a heart-beat rate and to increase the estimate of the energy production of the aerobic system and decrease the estimates of the energy productions of the phosphagen system and the glycolytic system in response to an increase of the heart-beat rate.
